(19) United States
(12) Patent Application Publication
(10) Pub. No.: US 2008/0152614 A1
(43) Pub. Date: Jun. 26, 2008

(54) METHOD AND KIT FOR SKIN LESION PREVENTION AND/OR PROTECTION

(75) Inventor: Wayne K. Dunshee, Maplewood, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/615,648

(22) Filed: Dec. 22, 2006

(51) Int. Cl. A61K 31/785

(52) U.S. Cl. 424/78.07; 424/78.02

(57) ABSTRACT

A method for treatment or prevention of skin lesions, e.g., blisters, comprising: a) applying a liquid polymerizable foundation composition to a selected location on skin, e.g., an existing lesion or an area to be protected from formation of a lesion, b) at least partially polymerizing the foundation composition in situ to form a foundation layer, and c) applying a liquid cap formulation to the foundation layer. Also a kit for carrying out such method.

### FIELD

A method for preventing or protecting skin lesions such as blisters by forming protective coatings is disclosed. Also, a kit for forming such protective coatings.

### BACKGROUND

Blistering of the skin typically is caused by some type of trauma. One example of such trauma is friction between an item of clothing or a shoe, and the skin surface beneath the clothing or shoe. Another example of such trauma is scalding or burning of the skin by a heated or cold object or fluid. Blisters may form as a result of, for example, a second-degree burn caused by scalding water or gas, or contact with a hot surface. Another example of trauma is an infection or disease such as pemphigus. Such trauma causes fluid to collect in a blister structure either under the epidermis or within the epidermis. This fluid is sterile, and protects the underlying dermal layer from pathogens. Preferably, the blister structure is prevented from popping until healing has occurred to maintain protection of the underlying dermal layer. Once healing has occurred, the fluid within the blister is reabsorbed into the body, and the overlying, dead tissue may either fall away or, if necessary, be debrided. Regardless of the cause of the blister, a blister may be popped as a result of frictional forces that bear on the blister, puncture from a sharp instrument or the application of pressure to the blister.

Typically, blisters or wounds are covered with bandages or the like to prevent direct frictional contact between clothing or other surfaces and the blister or wound, or to prevent sharp instruments from puncturing the blister. Such conventional bandages generally include a sterilized pad centrally mounted on a relatively larger adhesive strip such that the adhesive strip extends past the sterilized pad on either side, or all around the perimeter thereof. When the sterilized pad is placed over a blister, the extended portion of the adhesive strip adheres to the skin adjacent to the blister to maintain the pad in place. However, with many such conventional bandages, friction applied to the bandage by clothing or the like may be transferred to the surface of the blister, causing it to pop or to a wound, causing further damage to the wound.

Chaffing and blisters that result from frictional interaction between clothing or footwear and the skin may be prevented if such friction is reduced or prevented. Bandages have been proposed to prevent chaffing and the formation of blisters or other damage to the skin caused by such frictional forces. Conventional bandages used for this purpose typically are ineffectual, for the reasons discussed above, since the bandage itself will be caused to rub against the skin by the clothing or the footwear, and this rubbing also can cause blisters or other damage.

The ineffectiveness of conventional bandages for preventing chaffing, blisters or other injury or for protecting wounds or blisters is particularly acute with respect to blisters caused by ill-fitting footwear in which a portion of the footwear repeatedly rubs against the skin. This repeated rubbing produces and maintains the blister each time the footwear is worn. Such rubbing forces imposed on bandages, whether compressive or shear forces, often are transmitted to the inner protective layer of the bandage and to the underlying skin. Those forces either injure the area or, if it is injured already, prevent the area from properly healing.

Although bandages for preventing chaffing, protecting blisters and absorbing forces are known, there is a need for improved methods and structures that are more effective in shielding an injured area of the skin and preventing injury.

### SUMMARY

In one aspect, the present invention provides a method for preventing or treating skin lesions such as blisters that is both more convenient and more effective than prior methods. In brief summary, the method of the invention comprises: a) applying a liquid polymerizable foundation composition to a selected location on skin, e.g., an existing lesion or an area to be protected from formation of a lesion, b) at least partially polymerizing the foundation composition in situ to form a foundation layer, and c) applying a liquid cap formulation to the foundation layer.

In another aspect, the present invention provides kits for treating skin in accordance with the method of the invention. Briefly summarizing, kits of the invention comprise a dispensible liquid polymerizable foundation composition and a dispensible liquid cap formulation.

The method and kit of the invention provides improved, convenient treatment of injured areas of skin and means for preventing injury.

### DETAILED DESCRIPTION OF ILLUSTRATIVE EMBODIMENTS

As described above, in brief summary the method of the invention comprises: a) applying a polymerizable foundation composition to a selected location on skin, b) at least partially polymerizing the foundation composition in situ to form a foundation layer, and c) applying a cap formulation over the foundation layer to form a protective cap layer thereover. Collectively, the foundation layer and cap layer function as a protective membrane over the affected skin. The membrane is breathable yet inhibitive to penetration of germs and external contaminants, thereby facilitating healing of underlying skin lesions

The invention may be used to provide a protective membrane over a selected location on skin, e.g., an existing lesion or a location at which a lesion has not yet formed but which is considered at risk.

Once applied, there is typically no need to effect purposeful removal of the protective membrane or to cause the skin trauma potentially associated with such removal. The membrane forms a bacteriostatic or bactericidal barrier to external sources of contamination as well as barrier to fluid loss through the lesion. Moreover, in a preferred embodiment, the foundation composition is formulated to contain an antimicrobial agent which, over time, will be released from the resulting film thereby providing for protection against infection.

Foundation Layer

The foundation layer can be conveniently formed using so-called liquid bandage compositions, e.g., cyanoacrylate prepolymer compositions. The term “polymerizable cyanoacrylate esters” refers to polymerizable formulations comprising cyanoacrylate monomers or polymerizable oligomers which, in their monomeric form, are preferably compounds represented by formula I as described below.

The cyanoacrylate prepolymer composition can be applied as a liquid/gel to the desired location on the skin surface. If desired, it can include therapeutic agents such as analgesics, anti-inflammatory agents, antimicrobial agents, etc.

Preferably, the polymerizable cyanoacrylate ester comprises an ester which, in monomeric form, is represented by formula (I):

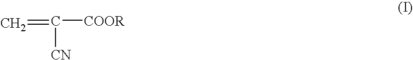

wherein R is selected from the group consisting of alkyls of 1 to 10 carbon atoms, alkenyls of 2 to 10 carbon atoms, cycloalkyl groups of from 5 to 8 carbon atoms, phenyl, 2-ethoxyethyl, 3-methoxybutyl, and a substituent of the formula (II):

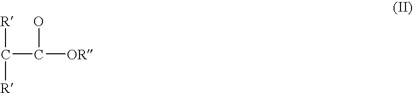

wherein each R′ is independently selected from the group consisting of: hydrogen and methyl, and R″ is selected from the group consisting of alkyls of from 1 to 6 carbon atoms, alkenyls of from 2 to 6 carbon atoms, alkynyls of from 2 to 6 carbon atoms, cycloalkyls of from 3 to 8 carbon atoms, aralkyls selected from the group consisting of benzyl, methylbenzyl, and phenylethyl, phenyl, and phenyl substituted with 1 to 3 substituents selected from the group consisting of hydroxy, chloro, bromo, nitro, alkyl of 1 to 4 carbon atoms, and alkoxy of from 1 to 4 carbon atoms.

More preferably, in the cyanoacrylate esters of formula I, R is alkyl of from 2 to 10 carbon atoms and more preferably alkyl of from 2 to 8 carbon atoms. Even more preferably, R is butyl, pentyl or octyl and most preferably, R is n-butyl.

More preferably, in formula I, R is an alkyl group of from 2 to 10 carbon atoms including ethyl, n-propyl, iso-propyl, n-butyl, iso-butyl, sec-butyl, n-pentyl, iso-pentyl, n-hexyl, iso-hexyl, 2-ethylhexyl, n-heptyl, octyl, nonyl, and decyl. More preferably, R is butyl, pentyl or octyl and most preferably, R is n-butyl. Mixtures of such compounds can also be employed as disclosed by U.S. Pat. No. 5,998,472 (Berger et al.) which is incorporated herein by reference in its entirety.

A preferred cyanoacrylate ester for use in the invention is n-butyl-2-cyanoacrylate.

The polymerizable cyanoacrylate esters described herein rapidly polymerize in the presence of water vapor or tissue protein, and the n-butyl-cyanoacrylate bonds to mammalian skin tissue without causing histotoxicity or cytotoxicity.

Such polymerizable cyanoacrylate esters are sometimes referred to herein as prepolymers and compositions comprising such esters are sometimes referred to herein as prepolymer compositions.

Several suitable polymerizable cyanoacrylate esters are known in the art and are described in, for example, U.S. Pat. Nos. 3,527,224; 3,591,676; 3,667,472; 3,995,641; 4,035,334; and 4,650,826 the disclosures of each are incorporated herein by reference in their entirety.

The treatment protocol may involve skin preparation prior to in situ formation of the foundation layer. Specifically, if a blister has formed, the site is conventionally treated by the attending health care professional by cleaning with an appropriate antimicrobial composition. The lesion site is preferably dried, e.g., blotted dry, and then an adherent polymeric film is formed over this site by applying a cyanoacrylate composition and permitting it to polymerize. As noted above, this composition comprises polymerizable cyanoacrylate monomers and/or reactive oligomers which, upon contact with the skin polymerize in situ to form a polymeric film. If a blister has already formed, the composition will cauterize the wound.

Polymerization occurs at ambient conditions for a sufficient period of time to allow polymerization to proceed. In general, the particular length of time required for polymerization will vary depending on factors such as the amount of adhesive composition applied, the temperature of the site, the moisture content of the site, the surface area of site, and the like. However, in a preferred embodiment, polymerization is generally complete within about 10 to about 60 seconds while the site is maintained at ambient conditions; however, in some cases, polymerization can occur up to about 5 minutes. During this period, the patient is maintained in a position which permits the cyanoacrylate to polymerize and form a polymeric film while minimizing any patient movement which might dislodge the cyanoacrylate from that site or create undesirable bonding.

Sufficient amounts of the composition are employed to cover (i.e., coat) the entire lesion site with a layer of the cyanoacrylate polymer. It is typically preferred that the resultant polymerized cyanoacrylate composition have a thickness of from about 0.5 to about 2 mills. The resultant layer preferably covers at least the entire area of the skin lesion. If necessary, excess cyanoacrylate monomer and/or oligomer can be removed with a wipe or tissue paper before polymerization or, after polymerization, any polymer formed at unintended sites can be removed with materials such as acetone. Application of a suitable amount of cyanoacrylate composition to a unit area to obtain desired thicknesses and coverage is well within the skill of the art.

The polymerizable cyanoacrylate composition can applied from a single dose product or by use of a multiple use dispenser which governs the amount of material applied onto a unit area of surface skin. In this regard, the dispenser described in U.S. Pat. No. 4,958,748 (Otake), which is incorporated by reference in its entirety, is one example of a dispenser which dispenses the cyanoacrylate adhesive composition in a controlled dropwise manner. Other methods for the controlled dispersement of the cyanoacrylate adhesive include, by way of example, a spray applicator, brush, wipe, swab or solid paddle applicator, applicators for repeated and intermittent use of the cyanoacrylate composition and the like.

In applicators, the cyanoacrylate composition can be conveniently stored at ambient conditions and can be provided in sterile form.

The cyanoacrylate compositions comprising the polymerizable cyanoacrylate esters are prepared by conventional methods of mixing the appropriate components until homogenous.

The desired specific viscosity of the foundation composition (and the cap formulation as described below) depends, in part, on the intended application. For example, relatively low viscosities are often preferred where application is to be made to a large surface area (e.g., abdominal surfaces). This preference results from the fact that those forms are less viscous and, accordingly, will permit more facile large surface area application of a thin application. Contrarily, where application is to be made to a specific position on the skin (e.g., elbow surfaces, knee surfaces and the like), higher viscosity compositions, including those containing thixotropic materials, are preferred to prevent “running” of the compositions to unintended locations.

Accordingly, these compositions have a viscosity of from about 2 to about 50,000 centipoise at 20° C. Preferably the less viscous compositions have a viscosity of from about 2 to about 1,500 centipoise at 20° C. More preferably, the cyanoacrylate ester employed in these compositions is almost entirely in monomeric form and the composition has a viscosity of from about 5 to about 500 centipoise at 20° C.

A thickening agent is optionally employed to increase the viscosity of the composition, which thickening agent is any biocompatible material which increases the viscosity of the composition. Suitable thickening agents include, by way of example, polymethyl methacrylate (PMMA) or other preformed polymers soluble or dispersible in the composition, a suspending agent such as fumed silica and the like with PMMA being preferred. Fumed silica is particularly useful in producing a gel for topical application having a viscosity of from about 1500 to about 50,000 centipoise at 20° C. Suitable thickening agents for the compositions described herein also include a partial polymer of the alkyl cyanoacrylate as disclosed in U.S. Pat. Nos. 3,654,239 and 4,038,345 both of which are incorporated herein by reference in their entirety.

Thickening agents are deemed to be biocompatible if they are soluble or dispersible in the composition and are compatible with the skin as measured by the lack of moderate to severe skin irritation.

If desired, the foundation layer composition may comprise other adjuvants to impart or improve certain properties as desired. Any adjuvants should be compatible with the polymerizable foundation composition, i.e., not unduly impair its shelf life or package stability and not unduly interfere with the ability of the composition to polymerize in situ in accordance with the present invention.

An illustrative adjuvant is, a “biocompatible plasticizer”, i.e., a material which is soluble or dispersible in the cyanoacrylate composition, which increases the flexibility of the resulting polymeric film coating on the skin surface, and which, in the amounts employed, is compatible with the skin as measured by the lack of moderate to severe skin irritation may be incorporated. Suitable plasticizers are well known in the art and include those disclosed in U.S. Pat. Nos. 2,784,127 and 4,444,933 the disclosures of both of which are incorporated herein by reference in their entirety. Specific plasticizers include, by way of example only, acetyl tri-n-butyl citrate (preferably about 20 weight percent or less), acetyl trihexyl citrate (preferably about 20 weight percent or less) butyl benzyl phthalate, dibutyl phthalate, dioctylphthalate, n-butyryl tri-n-hexyl citrate, diethylene glycol dibenzoate (preferably about 20 weight percent or less) and the like. The particular biocompatible plasticizer employed is not critical and preferred plasticizers include dioctylphthalate and C₂ to C₄ -acyl tri-n-hexyl citrates.

Another illustrative adjuvant is an “antimicrobial agent”, i.e., an agent which destroys microbes (i.e., bacteria, fungi, yeasts, and viruses) thereby preventing their development and their pathogenic action.

The cyanoacrylate adhesive compositions preferably include a biocompatible plasticizer and such plasticizers are preferably included in the composition from about 10 to about 30 weight percent and more preferably from about 18 to about 25 weight percent based on the total weight of the composition absent any antimicrobial agent.

Additionally, the cyanoacrylate compositions described herein preferably include a polymerization inhibitor in an effective amount to inhibit premature polymerization of the composition during storage. In a particularly preferred embodiment, this inhibitor is sulfur dioxide which is employed at from about 50 to about 500 ppm, preferably about 200 to about 500 ppm, based on the total weight of the composition absent any antimicrobial agent. Other preferred polymerization inhibitors include glacial acetic acid, free radical inhibitors (e.g., hydroquinones) and the like which can be used alone or in combination with SO₂.

Preferred cyanoacrylate compositions useful in the practice of this invention are also disclosed by U.S. Pat. No. 5,480,935 (Greff et al.), which application is incorporated herein by reference in its entirety. In a particularly preferred embodiment, the cyanoacrylate adhesive composition further comprises an antimicrobially effective amount of a compatible antimicrobial agent. Such compositions preferably comprise from about 1 to about 40 and preferably 5 to 30 weight percent of the compatible antimicrobial agent either as a solution or as a suspension based on the total weight of the composition. Compatible antimicrobial agents are those which are either soluble or suspendable in the cyanoacrylate composition, which do not cause premature polymerization of the cyanoacrylate composition, which do not prevent polymerization of the cyanoacrylate composition when applied to mammalian skin, and which are compatible with the intended use including biocompatibility with the patient's skin.

In a particularly preferred embodiment, the compatible antimicrobial agent comprises a complex of iodine molecules with a biocompatible polymer. Such complexes are well known in the art and the resulting complex typically comprises both available iodine and iodide anions. These complexes, on contact with mammalian skin, provide for a source of antimicrobial iodine. In any event, such complexes are employed only as starting materials herein and, by themselves, do not form a part of this invention. Suitable biocompatible polymers include, by way of example only, polyvinylpyrrolidone polymer which, when complexed with iodine, is also referred to under the common name of povidone-iodine available from BASF, Mt. Olive, N.J. When povidone-iodine is employed in the cyanoacrylate composition, it is preferably from about 5 to about 40 weight percent and more preferably from about 10 to about 25 weight percent is added to the cyanoacrylate composition based on the total weight of the composition.

Other suitable antimicrobial agents include complexes of iodine molecules with copolymers of vinylpyrrolidone and vinyl acetate, copolymers of vinylpyrrolidone and vinyl acetate cross-linked with polyisocyanates, copolymers of vinylpyrrolidone and vinyl functionalities, polymers of pyrrolidone and the like. Preferably, however, the iodine containing polymer is Povidone Iodine which is commercially available from a number of sources.

The use of a compatible antimicrobial agent in the composition permits the agent to be released from the polymeric film thereby reducing microbial growth under the film.

Other medicaments suitable for use in conjunction with the cyanoacrylate composition include corticoid steroids such as described by in U.S. Pat. No. 5,962,010 (Greff et al.) which is incorporated herein by reference in its entirety and analgesic compounds such as lidocaine. The former reduces inflammation at the site of the lesion whereas the latter reduces pain. Combinations of a steroid with an analgesic are also covered.

Cap Layer

Once the foundation layer is formed, a cap is formed thereover. The cap layer provides protection of the foundation layer and underlying skin lesion, if any. The foundation layer serves to seal or cauterize the skin lesion, and provide a suitable substrate for the cap layer. Many of the cap layer compositions described below would not exhibit desired stability on an active skin lesion.

The cap layer can be formed from such compositions as the coating compositions disclosed in U.S. Pat. No. 6,383,502 (Dunshee et al.) which is incorporated herein by reference in its entirety.

An illustrative composition comprises from about 1 to about 40% siloxane-containing polymer; about 60 to about 99% of an Alkane-Based Siloxy Polymer Reaction Solvent, and 0 to about 15% of adjuvants.

A method of making a siloxane-containing polymer is also provided comprising vinyl containing alkylsiloxysilanes alone or as co-, ter- or multi component polymers including other polymerizable monomers, which method comprises undertaking the polymerization in an alkane solvent selected from the group consisting of C₅O₅ to C₉ straight, branched, or cyclic alkanes so that the reaction provides a composition having a polymer content greater than about 15% by weight.

In some embodiments of the invention, the solvent system is selected such that it is an Alkane-Based Siloxy Polymer Reaction Solvent. An Alkane-Based Siloxy Polymer Reaction Solvent is a solvent system that primarily contains straight, branched or cyclic alkanes, and is capable of acting as the reaction solvent (i.e., the non-reactive fluid portion of a reaction composition) for the polymerization reaction of the specific monomer composition of TRIS/Methyl Methacrylate/2-Ethylhexyl acrylate in a 53/39/8 weight ratio. The solvent system is readily identified in a routine evaluation by undertaking a polymerization reaction using the specific monomer composition described above under a Standard Polymerization Reaction as defined below.

A Standard Polymerization Reaction comprises reacting 20% total monomer concentration by weight based on monomer plus solvent with VAZO 67 free radical initiator used at 0.3% by weight based on total monomer at a reaction temperature of 70° C. under nitrogen for 36 hours (or less time if greater than 90% monomer conversion has occurred). A solvent system is deemed to be an Alkane-Based Siloxy Polymer Reaction Solvent if, after cooling to room temperature, the reacted composition yields a clear, pourable solution of polymer, and the inherent viscosity (“IV” as tested by ASTM D2857-95 at 25° C. and according to principles discussed in Experiments in Polymer Science, by Edward A. Collins, Jan Bares and Fred W. Billmeyer, New York, Wiley (1973) pp 146-153.) of the polymer product is measured in ethyl acetate solvent at a nominal solids concentration of 0.5% (w/v)is less than 0.5 dl/g. Solvents which yield polymer with inherent viscosity greater than 0.5 dl/g are unsuitable.

A specific procedure for the test is detailed below.

A mixture of 4.24 g TRIS, 3.12 g methyl methacrylate and 0.64 g 2-ethyl hexyl acrylate is dissolved in 32 g of solvent in a 4 oz narrow mouth flint glass bottle and 0.024 g of VAZO 67 is added. The solution is purged with nitrogen at a flow rate of 1 liter/ minute for two minutes to remove dissolved oxygen. The bottle is closed tightly with a teflon lined metal cap and placed in a launder-o-meter preset at 70° C. for at least 24 hours. Conversion is determined from measurement of percent non volatile solids by loss on drying at 105° C. for 60 minutes.

Preferred solvents of the present invention are selected from one or more C₅ to C₁₂ straight, branched, or cyclic alkanes. Particularly preferred solvents are methylcyclopentane; n-heptane; n-octane; n-nonane; 2,2,4-trimethyl pentane; 3,4-dimethyl hexane. The solvent system may also comprise a blend of solvents that are a mixture of straight, branched or cyclic C₁₀ to C₁₂ alkanes with one or more C₅ to C₉ straight, branched, or cyclic alkanes.

For example, preferred solvent blends include mixtures of one or more of n-decane, n-undecane or n-dodecane with one or more of methylcyclopentane; n-heptane; n-octane; n-nonane; 2,2,4-trimethyl pentane; 3,4-dimethyl hexane.

The present inventive compositions are preferably provided as a skin protecting conformable bandage that is painted on. Alternatively, the compositions of the present invention may be provided as a component in a cosmetic or medicament containing composition.

The liquid polymer-containing coating materials of this invention comprise a siloxane containing polymer and a solvent system which is non-stinging to a user. Preferably the polymer is present from about 1 to about 40% by weight and the solvent is present in amounts of about 60 to about 99%. The material forms a coating or bandage in the form of a dried film when applied to a surface or the skin of a user.

Advantageously, the present invention provides the ability to manufacture the polymer in the same solvent as used in the final formulation. It has been found that it is exceedingly difficult to manufacture this polymer in the prior art solvent system hexamethyl disiloxane (“HMDS”). The ability to manufacture in the same solvent as the ultimate product is a significant advantage in cost savings, and additionally provides a more complete distribution of reaction products that may aid in film formation.

Use of the solvent system of the present invention allows for incorporation of higher solids content of the polymer, and also allows for selection of the polymer formulation to provide suitable materials for the desired use at lower siloxy silane component content.

It has surprisingly been found that the alkane solvents as described herein may be highly effective cosolvents with volatile siloxane (such as HMDS), which can substantially increase the amount of solids content that may be obtained without becoming pituitous. Additionally, it has been found that the addition of about 5% to about 10% of Tea Tree Oil (oil of Melaleuca alternifolia) and the like similarly increases the amount of solids content that may be obtained without becoming pituitous.

Preferably, the siloxane containing polymer comprises at least one vinyl containing alkylsiloxysilane and an addition polymerizable comonomer.

It is a feature of the invention that the liquid materials can act at room temperature (20° C.) when applied to skin, nails, or mucous membranes of a user to form films in minutes or less, which films are excellent bandages. The films are conformable, comfortable and can be elastic and flexible. The films do not irritate the skin and mucous membrane when sprayed or deposited in any way during application and in use after drying. The bandages are substantially painless and can be easily removed substantially without pain. The dried bandages formed are substantially non-water sensitive, and waterproof and have high water vapor and oxygen gas transmission therethrough. The bandages form when applied over surfaces wet with water, blood or body fluids, in short times at standard room temperature and reasonable variants thereof. The liquid composition and/or dried polymer film can have various medicaments or other agents incorporated therein for maintaining sterility and/or for release to the underlying area of the body of a user. For example, perfumes, antimicrobial, botanicals, medicants, or similar materials can be released from the coatings.

The siloxane containing polymers of this invention can comprise vinyl containing alkylsiloxysilanes alone or as co-, ter- or multi-component polymers which can include other polymerizable monomers that do not make the polymers hydrophilic.

Typical vinylaklylsiloxysilanes that may be utilized are:

3-methacryloyloxypropyltris(trimethylsiloxy)silane (TRIS);

3-methacryloyloxypropylpentamethyldisiloxane;

3-methacryloyloxypropylbis(trimethylsiloxy)methylsilane;

3-acryloyloxypropylmethylbis(trimethylsiloxy)silane;

3-acryloyloxypropyltris(trimethylsiloxy)silane; and others.

Typical addition polymerizable monomers which may be reacted with the vinylalkylsiloxysilanes to form multi component polymers are: methyl methacrylate methyl acrylate, tetrahydrofurfuryl methacrylate, cyclohexyl acrylate, tetrahydrofurfuryl acrylate, n-lauryl acrylate, n-lauryl methacrylate, 2-phenoxyethyl acrylate, 2-phenoxyethyl methacrylate, isodecyl acrylate, isodecyl methacrylate, isooctyl acrylate, isooctyl methacrylate, isobornyl acrylate, isobornyl methacrylate, benzyl methacrylate, 2-butoxyethyl acrylate, n-butyl acrylate, n-butyl methacrylate, ethyl acrylate, ethyl methacrylate, dimethyl itaconate, di-n butyl itaconate, 2-ethylhexyl acrylate, 2-ethylhexyl methacrylate, furfuryl methacrylate, n-hexyl acrylate, n-hexyl methacrylate, isobutyl acrylate, isobutyl methacrylate, isopropyl methacrylate, methyl acrylate, alpha methyl styrene, styrene, p-t-butyl styrene, 4-methoxystyrene, n-octadecyl acrylate, n-otadecyl methacrylate, 2-phenylethyl methacrylate, n-tridecyl methacrylate, vinyl benzoate, vinyl naphthalene. In addition, fluorinated siloxanes, fluorinated itaconates, fluorinated methacrylates or acrylates, such as hexafluoroisopropyl methacrylate, can be used.

Any hydrophobic polymerizable monomer can be used as long as the resulting copolymer has desired O₂ and H₂O vapor permeability. These additional polymerizable comonomers can be present in amounts up to about 0.85 mole fraction.

The polymers of the invention are preferably in proportions from about 15 to about 100 mole % vinylalkylsiloxysilane which component maintains the desired compatibility of the polymer in the volatile liquid polydimethylsiloxanes with polar adjuvants, provides high moisture and oxygen permeability, and provides biocompatibility. A range of about 20 to about 40 mole % of the vinylalkylsiloxysilane in the polymer is preferred in the polymer of this invention. Other addition polymerizable monomers may be copolymerized with the vinylalkylsiloxysilanes between from 0 to about 85% mole of the polymer composition to adjust permeability, adhesion, toughness, elasticity, temperature stability, and impact resistance, among other film qualities.

The polymers may be linear, branched, or slightly cross-linked and can be homo, co-, ter- or multi polymers. They may be random copolymers or segmental in nature.

Typical vinylalkylsiloxysilane monomers can have the following formulas:

CH₂═C(R¹)COOR²SiR³R⁴R⁵

where R¹ is H, CH₃, or CH₂COOR⁶; R² is alkyl (C₁ to C₄) or CH₂CH(OH)CH₂; where R³, R⁴, R⁵ is OSi(Y)₃, or alkyl (C₁ to C₆), and at least one of R³, R⁴, R⁵ is OSi(Y)₃, Y is alkyl (C₁ to C₆), OSi(Z)3, or R²OOC(R¹)C═CH₂, Z is alkyl (C₁ to C₆), or aryl; and R⁶ is R²SiR³R⁴R⁵

The polymers may have molecular weights from 50,000 to several million. The preferred molecular weight range is about 50,000 to about 500,000 weight average molecular weight. Lower molecular weight polymers have notably higher solubility in the solvents and solvent systems of this invention and hence, while they can be film formers, they generally are slow to dry and remain tacky. The molecular weight of the polymers may be controlled by varying initiator, initiator concentration, reaction temperature, reaction solvent, and/or reaction method.

Most preferably, the polymers of the invention are acrylate or methacrylate terpolymers having an A monomer component that is a silane derivative, a B monomer component that when provided as a homopolymer would prepare a “hard” polymer, and a C monomer component that, when provided as a homopolymer would prepare a “soft” polymer.

For the A monomer, examples of the silane derivatives are as described above. B monomers are “hard” where the corresponding homopolymer typically has a glass transition temperature (T_{g}) of more than about −5° C. Examples of such monomers are acrylate or methacrylate monomers, preferably C₁ to C₄ alkyl methacrylates. Most preferably, the hard monomer is methyl methacrylate.

Other examples of monomers that can be used for the hard monomer component are monomers having the requisite T_{g} values including methacrylates having a structure other than delineated above, such as benzyl methacrylate and isobornyl methacrylate methcrylamide such as N t butylmethacrylamide; acrylates such as isobornyl acrylate; acrylamides such as N butylacrylamide and N-t butylacrylamide; diesters of unsaturated dicarboxylic acids such as diethyl itaconate and diethyl fumarate; vinyl nitrites such acrylonitrile and methacrylonitrile; vinyl esters such as vinyl acetate and vinyl propionate; and monomers containing an aromatic ring such as styrene; .alpha.-methyl styrene and vinyl toluene. ‘C ’ monomers may be selected from monomers that form soft homopolymers. “Soft” monomers are monomers where the corresponding homopolymer typically has a Tg of less than about 10° C., provided that the hard monomer has a higher T_{g} than the soft monomer in each polymer. Such monomers are C₄ to C₁₂ alkyl acrylates and C₆ to C₁₂ alkyl methacrylates, wherein the alkyl groups are straight, branched, or cyclic. Most preferably, the soft monomer is selected from C₇ to C₁₀ straight chain alkyl acrylates.

Other examples of monomers that can be used for the soft monomer component are monomers having the requisite T_{g} values including dienes, such as butadiene and isoprene; acrylamides, such as N-octylacrylamide; vinyl ethers such as butoxyethylene, propyloxyethylene and octyl oxyethylene; vinyl halides, such as 1,1-dichloroethylene; and vinyl esters such as vinyl caprate and vinyl laurate.

It has been found that this mix of monomers provide particularly advantageous abilities to adjust mole fraction ratios to optimize oxygen permeability, ductility, moisture vapor transmissibility of the film and cost of materials. Highly durable coatings are particularly desired to enable the coating to remain on the skin for an extended time and to provide superior protection.

Most preferably, the siloxane-containing polymer comprises from about 50 to about 60 weight percent A monomer, from about 25 to about 45 weight percent B monomer, and about 3 to about 20 weight percent of C monomer. A specifically preferred embodiment is where the siloxane-containing polymer comprises about 50 to about 60 weight percent of 3-methacryloxypropyl tris(trimethylsiloxy)silane, about 25 to about 45 weight percent methyl methacrylate, and about 3 to about 20 weight percent of a monomer selected from C₇ to C₁₀ straight chain or branched alkyl acylates.

Particularly preferred siloxane-containing polymers have an overall effective T_{g} from about 20 to about 80° C., more preferably from about 40 to about 70° C., and most preferably from about 50 to about 60° C.

One variation in selection of monomers to be used in siloxane containing polymer is using more than one monomer within each category A, B, or C. For example, the polymer could comprise 57% 3-methacryloyloxypropyl tris(trimethylsiloxy)silane, 39% methyl methacrylate, 2% isooctyl acrylate and 2% decyl acrylate. The last two monomers each satisfy the definition of the C monomer, and together provide the desired quantity of this component.

Any free radical initiator can be used in forming the polymers including azobisisobutyronitrile; 2,2′-azobis(2,4 dimethylpentane nitrile); 2,2′-azobis-(2-methylbutane nitrile); potassium persulfate; ammonium persulfate; benzoyl peroxide; 2,5-dimethyl 2,5-bis(2-ethylhexanoylperoxy)hexane; and the like. The polymerization can be carried out by solution, emulsion, or suspension techniques.

Preferably, a polymer comprised of methyl methacrylate, isooctyl acrylate, and (“3-methacryloxypropyl tris(trimethylsiloxy)silane) (“TRIS”) is polymerized directly in a composition of 90% 2,2,4-trimethylpentane with 10% 3,4-dimethylhexane (Permethyl.RTM. 97A from Permethyl Specialties, LLC, Milmay, N.J.) or n-heptane at 25% nonvolatile finished polymer.

Adjuvants of the present compositions may comprise cosolvents, suspending aids, preservatives, antioxidants, active ingredients such as medicaments, humectants, emollients, slipagents, waxes, colorants (including dyes, pigments, colored particles, glitter and the like), flavorants, fragrances and the like. Adjuvants may also include solid materials, such as titanium dioxide and silica. Such materials may reduce tackiness of the overall composition, and additionally may perform a function such as acting as a sunscreen, handling modifier or to modify composition drying rates.

Preferred cosolvents that may be used as adjuvants of the present compositions include alcohols, ketones, oils and the like. Preferably, these cosolvents are present in an amount such that they do not render the overall composition to have a stinging effect upon application to the skin.

The high solids polymer is compatible with a variety of useful adjuvants such as aloe vera in mineral oil, vitamin E, vitamin A, palmitate, triclosan, methyl salicylate, menthol, capsicum oleoresin, tea tree oil, squalane, peppermint, citronella, spearmint, jojoba oil, sweet almond oil, other oil and oil soluble materials. Using oils with higher carbon chains will vary the evaporation rates and/or skin absorption rates and provide short term plasticizing of the polymer, which in turn gives the polymer coating a glossy visible appearance until the solvent evaporates and/or absorbs.

The compositions of this invention may be applied in liquid form by utilization of an applicator, such as a brush, rod, finger, sponge, cloth, dropper, etc; in spray or mist form; or any other usable technique for applying a liquid to a surface. The composition is preferably applied so as to at least cover the entirety of the previously formed foundation layer. Typically it will be preferred to apply cap composition to the entirety of the foundation layer and a surrounding perimeter.

Surprisingly, compositions of the present invention may be formulated to provide excellent sprayable siloxane-polymer containing compositions. A sprayable composition may surprisingly be provided having a polymer content of as high as about 6 to about 10% by weight.

Medicants may be incorporated into the liquid or solid, dried film bandages for ready or continual release as the invention provides for an inert, longlasting, highly permeable film which can contain medicant or other active agents to be applied to the skin, mucous membranes and other body areas on which it is desired to release the active agent over an extended period of time. Examples of useful medicants are fungicides, pesticides, antimicrobial agents, antiviral agents, antitumor agents, blood pressure and heart regulators, and many more. Other types of active agents which may be desirable to incorporate include perfumes, plant growth regulators, DEET, plant insecticides, UV, and IR absorbers, etc.

What is claimed is:

1. A method for treating or preventing a skin lesion comprising: a) applying a polymerizable foundation composition to a selected location on skin, b) at least partially polymerizing said composition in situ to form a foundation layer, and c) applying a cap formulation to said foundation layer.

2. The method of claim 1 wherein said polymerizable composition comprises a cyanoacrylate ester which in monomeric form is represented by formula I: 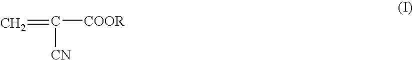
where R is selected from the group consisting of: alkyls of 1 to 10 carbon atoms, alkenyls of 2 to 10 carbon atoms, cycloalkyl groups of from 5 to 8 carbon atoms, phenyl, 2-ethoxyethyl, 3-methoxybutyl, and substituents of the formula (II): 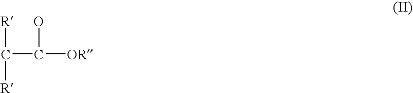
wherein each R′ is independently selected from the group consisting of: hydrogen and methyl, and R″ is selected from the group consisting of: alkyls of from 1 to 6 carbon atoms, alkenyls of from 2 to 6 carbon atoms, alkynyls of from 2 to 6 carbon atoms, cycloalkyls of from 3 to 8 carbon atoms, aralkyls selected from the group consisting of benzyl, methylbenzyl, and phenylethyl, phenyl, and phenyl substituted with 1 to 3 substituents selected from the group consisting of hydroxy, chloro, bromo, nitro, alkyl of 1 to 4 carbon atoms, and alkoxies of from 1 to 4 carbon atoms.

3. The method of claim 1 wherein said cap formulation comprises:
a) from about 1 to about 40% of siloxane-containing polymer comprising at least one vinyl containing alkylsiloxysilane and an addition polymerizable comonomer, wherein the vinyl containing alkylsiloxysilane has the following formula:
CH₂═C(R¹)COOR²SiR³R⁴R⁵
wherein R¹ is H, CH₃, or CH₂COOR⁶ where R⁶ is R²SiR³R⁴R⁵; R² is alkyl (C₁ to C₄) or CH₂CH(OH)CH₂; R³, R⁴, or R⁵ is OSi(Y)₃ or alkyl (C₁ to C₆) and at least one of R³, R⁴, or R⁵ is OSi(Y)₃; Y is CH₃ or OSi(Z)₃; and Z is CH₃;
b) from about 60 to about 99% of an Alkane-Based Siloxy Polymer Reaction Solvent; and
c) from 0 to about 15% of adjuvant;
wherein the composition is in the form of a solution.

4. The method of claim 3 wherein said Alkane-Based Siloxy Polymer Reaction Solvent is selected from the group consisting of C₅ to C₁₂ straight, branched, or cyclic alkanes.

5. The method of claim 3 wherein said Alkane-Based Siloxy Polymer Reaction Solvent is selected from the group consisting of hexane, heptane, octane, nonane, and mixtures thereof.

6. The method of claim 3 wherein said Alkane-Based Siloxy Polymer Reaction Solvent is a blend of a first solvent selected from the group consisting of hexane, heptane, octane, nonane and mixtures thereof and a second solvent selected from the group consisting of decane, undecane, dodecane, and mixtures thereof.

7. The method of claim 1 wherein at least one of said polymerizable foundation composition and said cap formulation further comprises an antimicrobially effective amount of a compatible antimicrobial agent.

8. The method of claim 7 wherein the compatible antimicrobial agent is polyvinylpyrrolidone iodine.

9. The method of claim 1 wherein at least one of said polymerizable foundation composition and said cap formulation further comprises a biocompatible plasticizer.

10. The method of claim 9 wherein said biocompatible plasticizer is dioctyl phthalate.

11. The method of claim 1 wherein said foundation composition further comprises a polymerization inhibitor.

12. The method of claim 11 wherein said polymerization inhibitor is SO₂.

13. The method of claim 1 further comprising renewing said cap layer by e) applying a quantity of cap formulation to a previously treated site, and d) drying such cap formulation to form a cap layer.

14. A kit for preventing or treating skin lesions comprising a dispensible polymerizable foundation composition and a dispensible cap formulation.

15. The kit of claim 14 wherein said polymerizable foundation composition comprises a cyanoacrylate ester which in monomeric form is represented by formula I: 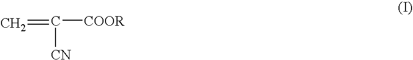
where R is selected from the group consisting of: alkyls of 1 to 10 carbon atoms, alkenyls of 2 to 10 carbon atoms, cycloalkyl groups of from 5 to 8 carbon atoms, phenyl, 2-ethoxyethyl, 3-methoxybutyl, and substituents of the formula (II): 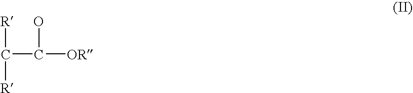
wherein each R′ is independently selected from the group consisting of: hydrogen and methyl, and R″ is selected from the group consisting of: alkyls of from 1 to 6 carbon atoms, alkenyls of from 2 to 6 carbon atoms, alkynyls of from 2 to 6 carbon atoms, cycloalkyls of from 3 to 8 carbon atoms, aralkyls selected from the group consisting of benzyl, methylbenzyl, and phenylethyl, phenyl, and phenyl substituted with 1 to 3 substituents selected from the group consisting of hydroxy, chloro, bromo, nitro, alkyl of 1 to 4 carbon atoms, and alkoxies of from 1 to 4 carbon atoms.

16. The kit of claim 14 wherein said cap formulation comprises:
a) from about 1 to about 40% of siloxane-containing polymer comprising at least one vinyl containing alkylsiloxysilane and an addition polymerizable comonomer, wherein the vinyl containing alkylsiloxysilane has the following formula:
CH₂═C(R¹)COOR²SiR³R⁴R⁵
wherein R¹ is H, CH₃, or CH₂COOR⁶ where R⁶ is R²SiR³R⁴R⁵; R² is alkyl (C₁ to C₄) or CH₂CH(OH)CH₂; R³, R⁴, or R⁵ is OSi(Y)₃ or alkyl (C₁ to C₆) and at least one of R³, R⁴, or R⁵ is OSi(Y)₃; Y is CH₃ or OSi(Z)₃; and Z is CH₃;
b) from about 60 to about 99% of an Alkane-Based Siloxy Polymer Reaction Solvent; and
c) from 0 to about 15% of adjuvant;
wherein the composition is in the form of a solution.
